(19) United States
(12) Patent Application Publication
(10) Pub. No.: US 2013/0030073 A1
(43) Pub. Date: Jan. 31, 2013

(54) AMINE-INITIATED POLYOLS FROM RENEWABLE RESOURCES AND PROCESSES FOR THEIR PRODUCTION AND USE

(71) Applicants: Don S. Wardius, Pittsburgh, PA (US); Steven L. Schilling, Pittsburgh, PA (US)

(72) Inventors: Don S. Wardius, Pittsburgh, PA (US); Steven L. Schilling, Pittsburgh, PA (US)

(73) Assignee: Bayer MaterialScience LLC, Pittsburgh, PA (US)

(21) Appl. No.: 13/190,648

(22) Filed: Jul. 26, 2011

(51) Int. Cl. C08G 18/00; C07C 211/51; C07C 229/00; C07C 209/46

(52) U.S. Cl. 521/164; 564/396; 564/305; 554/103

(57) ABSTRACT

Amine-initiated polyether polyols are made by reacting an amine adduct with a triglyceride in the presence of an alkylene oxide to obtain a polyol having a total renewables content of at least 20%. The polyols produced in this manner are particularly useful for the production of polyurethane and a polyisocyanurate foams

### BACKGROUND OF THE INVENTION

The present invention relates to amine-initiated polyether polyols produced in part from renewable resources, to a process for their production and to the use of these polyether polyols in the production of rigid polyurethane foams.

Polyether polyols are known to be useful in the production of rigid polyurethane and polyurethane-polyisocyanurate foams. In one of the most common methods for the production of these polyols, a polyhydric alcohol such as sucrose is reacted with an alkylene oxide such as ethylene oxide or propylene oxide in the presence of an alkaline catalyst such as potassium hydroxide. Prior to use in the production of foams, any alkaline catalyst present in the polyol must be neutralized and/or removed to ensure that the catalyst will not interfere with the reaction between polyol and another reactive material such as a polyisocyanate. This is generally accomplished by addition of an acid to neutralize the alkaline catalyst. This neutralization frequently results in the formation of a solid salt in the polyol which salt may be removed by filtration. The removed solid is commonly called the filter cake.

One type of polyether polyol which has been found to be advantageous in foam-forming systems blown with non-CFC blowing agents is an amine-initiated polyether polyol. Such polyether polyols may be formed by reacting an amine such as toluene diamine with an alkylene oxide such as ethylene oxide or propylene oxide. These amine-based polyols are particularly advantageous for the production of rigid foams with non-CFC blowing agents which foams are characterized by low thermal conductivity, good dimensional stability, and good compressive strength. In addition, the amine-based polyols help promote the polyurethane reaction, which reduces the amount of catalyst that would otherwise need to be added.

Polyols derived from renewable resources are becoming more commercially desirable due partly to the increasing cost of petroleum-derived feedstocks. The “green” image of renewable-based products has also become a significant factor in the marketing of articles containing foam; however, the physical characteristics and properties are still required to be comparable to foams produced from petroleum-based polyols. In addition, retailers and government agencies are pushing for higher renewable resource content in the finished goods they sell or purchase to take advantage of this “green” image or to reduce the dependence on petroleum-based materials.

It would therefore be advantageous to develop an amine-based polyol produced, at least in part, from a renewable resource.

### SUMMARY OF THE INVENTION

It is an object of the present invention to provide an amine-initiated polyether polyol derived from renewable resources having an overall renewable content of from about 20 to about 85% by weight.

It is also an object of the present invention to provide a process for the production of an amine-initiated polyether polyol derived from renewable resources having an overall renewable content of from about 20 to about 85% by weight.

It is a further object of the present invention to provide amine-initiated polyether polyols derived from renewable resources which produce polyurethane foams having very good thermal insulation properties.

These and other objects which will be apparent to those skilled in the art are accomplished by reacting an amine, such as toluene diamine (TDA), with an epoxide such as ethylene oxide or propylene oxide to form an amine-based alkoxylated adduct. This amine-based adduct is then reacted with a triglyceride and optionally, a polysaccharide compound such as sucrose, in the presence of an alkylene oxide and a catalyst. Both the triglyceride and polysaccharide are renewable resources.

### DETAILED DESCRIPTION OF THE INVENTION

The present invention relates to amine-initiated polyether polyols which are derived from renewable resources, to a process for the production of these polyols and to rigid polyurethane foams produced from these polyols.

The polyols of the present invention are prepared from an amine-based adduct that is produced by reacting a diamine or polyamine with an alkylene oxide. This amine-based adduct is then reacted with a triglyceride and optionally, a polysaccharide compound like sucrose or sorbitol or a glycerol or a polyglycerol in the presence of an alkylene oxide and a catalyst. Methods for conducting such alkoxylation reactions are known in the art.

Generally, the polyol production process of the present invention is conducted at a temperature of from about 100 to about 150° C., preferably, about 125° C. The reaction vessel in which the polyol of the present invention is produced will generally be rated to hold at least 5 bar of pressure due to the need to contain the volatile alkylene oxide at the temperatures used to carry out the process of the present invention.

In general, the amine-based adduct used to produce the polyether polyols of the present invention is prepared by reacting an alkylene oxide with an amine having an amine functionality of at least 1, preferably, about 2. Generally, the amine and alkylene oxide are reacted in amounts such that from 1 to 2 moles of alkylene oxide, preferably, from 1.25 to 1.9 moles of alkylene oxide are present for each amine group. No added catalyst is required to carry out this initial reaction to form the amine-based adduct.

The amines useful in the production of the amine-based adduct have an average amine functionality of at least 1, preferably from about 2 to about 3 most preferably about 2. Examples of suitable amines include: aromatic amines such as crude toluene diamine obtained by the nitration of toluene followed by reduction; 2,3-toluene diamine; 3,4-toluene diamine; 2,4-toluene diamine; 2,6-toluene diamine; and isomeric mixtures of toluene diamine; aniline; 4,4′-methylene dianiline; methylene-bridged polyphenyl polyamines composed of isomers of methylene dianilines and triamines or polyamines of higher molecular weight prepared by methods known in the art; alkanol amines such as monoisopropanolamine, diisopropanolamine, monoethanolamine, diethanolamine organic amines such as ethylene diamine, diethylene triamine and the like; and Mannich reaction products of phenol or substituted phenols with alkanol amines and formaldehyde or paraformaldehyde. Mixtures of the above amines may also be used.

Examples of alkylene oxides useful in producing the amine-based adduct and useful in the reaction of the amine-based adduct with a triglyceride in the process of the present invention include: ethylene oxide, propylene oxide, butylene oxide, and mixtures of these alkylene oxides. Use of only ethylene oxide to produce the amine-based adduct is preferred. Ethylene oxide or propylene oxide or mixtures of ethylene oxide and propylene oxide are preferably included in the reaction of the amine-based adduct with the triglyceride.

The triglyceride(s) reacted with the amine-based adduct are generally reacted in amounts such that the weight ratio of triglyceride to amine is from about 3:1 to about 12:1, preferably, from about 4:1 to about 10:1, most preferably from about 5:1 to about 9:1.

Any of the naturally occurring plant oils, plant oil products, animal-derived fats or oils, synthetic triglycerides, heat or chemically treated triglycerides, modified triglycerides or epoxidized triglycerides may be used to produce the amine-initiated polyether polyols of the present invention. Examples of suitable triglycerides include: soybean oil, palm oil, palm kernel oil, castor oil, canola oil, high erucic acid content rapeseed oil, rapeseed oil, corn oil, jatropha oil, peanut oil, cottonseed oil, linseed oil, lard, tallow, bodied soybean oil, epoxidized soybean oil, camelina oil, lipids derived from algae, lesquerella oil, limnanthes (meadowfoam) oil and combinations thereof. In addition, it is possible to employ either crude or refined triglycerides in the polyol reaction process. Soybean oil (refined bleached de-odorized grade) is particularly preferred.

A naturally occurring sugar (i.e., a carbohydrate having at least one saccharose group) such as sucrose or sorbitol may optionally be included in the reaction mixture containing the amine-based adduct and the triglyceride(s). Glycerol or polyglycerol (such as Diglycerol™ by Solvay Chemicals) may also be used in addition to or in place of a sugar.

When a sugar is included in the reaction mixture composed of the amine-based adduct, triglyceride and alkylene oxide, it is generally included in an amount such that the molar ratio of sugar to triglyceride is from 0.01 to about 0.64, preferably, from about 0.2 to about 0.35.

When a glycerol is included in the reaction mixture composed of the amine-based adduct, triglyceride and alkylene oxide, it is generally included in an amount such that the molar ratio of glycerol to triglyceride is from 0.01 to about 0.5, preferably, from about 0.2 to about 0.4.

When a combination of sugar and glycerol is included in the reaction mixture composed of the amine-based adduct, triglyceride and alkylene oxide, the total amount of sugar and glycerol generally included is an amount such that the molar ratio of sugar plus glycerol to triglyceride is from 0.01 to about 0.55, preferably, from about 0.2 to about 0.44.

In principle, any alkaline material capable of catalyzing an epoxidation reaction may be used in the process of the present invention, particularly, during the reaction of the amine adduct with the triglyceride in the presence of an alkylene oxide. Specific alkaline catalysts which have been found to be particularly suitable include potassium hydroxide, sodium hydroxide, and amine catalysts such as imidazole or methyl-imidazole. Potassium hydroxide is particularly preferred. When used, potassium hydroxide is generally used in an amount that results in a concentration of from about 0.05 to about 0.5% after addition of the epoxide. It is preferred that any potassium hydroxide remaining in the reaction mixture after completion of the reaction be neutralized to promote stability of the polyol and ensure consistent performance in the intended applications. The potassium hydroxide catalyst can be neutralized with sulphuric acid to form potassium sulphate, which can be removed from the product by filtration. It can also be neutralize with acetic acid or lactic acid, to form salts which remain soluble in the product and do not need to be removed.

The amine-initiated polyether polyols of the present invention generally have a functionality of from about 1.5 to about 3.5, preferably from 1.7 to 3.2, most preferably, from about 2.0 to about 3.0, an equivalent weight (number average, determined by end group analysis) of from about 100 to about 600, preferably, from about 130 to about 500, most preferably, from about 150 to about 400, and an overall renewables content of from about 20 to about 85%, preferably, from about 50 to about 80%, most preferably, from about 65 to about 75%. These polyols generally have a viscosity at 25° C. of from about 100 to about 1500, preferably from about 150 to 800 mPa·sec.

The renewables content is calculated by a mass balance of the material charges to the reactor, followed by an assessment of the extent of the reaction by carrying out size exclusion chromatography of the reaction product. ASTM D6866 may be used to determine the biobased carbon content of the product.

After the polyol has been prepared, any residual catalyst remaining in the reaction mixture will generally be neutralized in accordance with techniques known to those skilled in the art. Neutralization need not be exact neutrality (i.e., pH=7.0). The reaction mixture may be maintained at a slight acidity or alkalinity, i.e., at a pH of from 5 to 11, preferably, from 6 to 10. It is preferred that any salt formed from the neutralized catalyst be soluble in the polyether polyol so that the product amine-initiated polyol may be used in polyurethane foam-forming equipment without subsequent treatment and without generating large amounts of solid waste material.

Examples of hydroxy carboxylic acids useful in neutralizing residual catalyst include: lactic acid, salicylic acid, substituted salicylic acids such as 2-hydroxy 3-methyl benzoic acid, 2-hydroxy 4-methyl benzoic acid and combinations of these acids. Lactic acid is particularly preferred.

The neutralized polyether polyol reaction mixture of the present invention is clear (i.e., free from haze) and may be used directly in processes for the production of polyurethane foams. Methods for the production of polyurethane foams from such polyether polyols are well known to those in the art.

Generally, a polyether polyol such as that produced in accordance with the present invention is reacted with an organic polyisocyanate in the presence of a blowing agent to produce a polyurethane foam.

Organic polyisocyanates which may be reacted with the amine-initiated polyether polyols of the present invention to produce good polyurethane foams include: 2,4-toluene diisocyanate, 2,6-toluene diisocyanate and isomeric mixtures of these diisocyanates; diphenylmethane-4,4′-diisocyanate diisocyanate and polymethylene polyphenyl polyisocyanates; 4,4′-methylene-bis-cyclohexyl diisocyanate; isophorone diisocyanate; and prepolymers of such polyisocyanates.

Blowing agents useful in the production of polyurethane foams from the amine-initiated polyether polyols of the present invention include: water; hydrochlorofluorocarbons such as 1,1-dichloro-1-fluoroethane (HCFC-141b), 1-chloro-1,1-difluoroethane (HCFC-142b), and chlorodifluoromethane (HCFC-22); hydrofluorocarbons such as 1,1,1,3,3-pentafluoropropane (HFC-245fa), 1,1,1,2-tetrafluoroethane (HFC-134a), and 1,1,1,4,4,4-hexafluorobutane (HFC-356mffm); halogenated olefins such as cis-1,1,1,4,4,4-hexafluorobutene (HFO-1336mzz-Z); the blowing agent available from Honeywell under the designation HBA-2; hydrocarbons such as isomers of pentane and cyclopentane; and mixtures of the above. Water, HFC-245fa, hydrocarbons and halogenated olefins or mixtures thereof are particularly preferred.

Other known auxiliary agents and additives such as catalysts, surfactants, stabilizers, emulsifiers, fillers, etc. may also optionally be included in foam-forming mixtures containing the amine-initiated polyether polyols of the present invention.

Any of the known methods for producing polyurethane foams may be used to produce foams from the amine-initiated polyether polyols of the present invention. These known methods include the one-shot process, a prepolymer process, and other processes.

The foams produced with the amine-based polyols of the present invention are characterized by properties comparable to those of foams produced with polyols derived from the traditional petroleum-based materials.

Having thus described our invention, the following Examples are given as being illustrative thereof. All of the parts and percentages given in these Examples are parts by weight and percentages by weight, unless otherwise indicated.

### EXAMPLES

The polyether polyols with high renewable contents described in Examples 1 and 2 were produced by a simultaneous transesterification alkoxylation process described more fully below. In each of Examples 1 and 2, potassium hydroxide (KOH) catalyst was employed to promote rapid and complete incorporation of all reactants. In Example 1, the KOH catalyst was used only in the final step.

### Example 1

### Preparation of NOP A (According to the Present Invention)

Ortho-toluene diamine (o-TDA) was treated to inhibit color formation in shipping and handling by addition of para-formaldehyde in accordance with the teachings of U.S. Pat. No. 6,004,482.

The o-TDA was first reacted with ethylene oxide using the inherent reactivity of the amine groups. 2635 gms of molten o-TDA were charged to a clean, dry stainless steel reactor. The reactor was closed, the atmosphere in the vessel was purged of air as completely as possible. Under a nitrogen atmosphere, the molten o-TDA was stirred and heat was applied to establish a uniform temperature of 130° C. A feed of ethylene oxide was initiated at a rate of 5 gm/min. This rate was gradually increased to 50 gm/min. A total of 3297 grams of EO was fed to the reactor in this manner. As the feed of ethylene oxide proceeded, the. temperature of the reactor was allowed to gradually increase to 150° C. After all of the ethylene oxide had been fed, the reaction mixture was allowed to post-react completely by holding the reaction mass at 150° C. with continued stirring for 1 hour. The resulting adduct of ethoxylated o-TDA was analyzed and was found to have a hydroxyl number of about 763 mg KOH/g.

2001 grams of the adduct of o-TDA, made as described above, were then charged to a clean, dry stainless steel reactor. 26.5 grams of 46% aqueous KOH were then added to the o-TDA adduct. 5255.4 grams of refined bleached de-odorized soybean oil (Cargill AR grade) were then added to the reactor. The reactor was closed and the atmosphere in the vessel was carefully purged of air. Under a nitrogen atmosphere, the reactor contents were stirred and heat was applied to establish a uniform temperature of 115° C. in the reactor. Then the contents of the reactor were carefully subjected to vacuum and gradual nitrogen sparging to the bottom portion of the liquids for one hour while maintaining the temperature at 115° C. in order to de-water the system. The vacuum was broken with nitrogen and the contents were gradually heated to establish a uniform temperature of 125° C. EO was then fed into the reactor at a rate of 5 gms/min. As the feed was established, the feed rate was gradually increased to 30 gm/min. A total of 533 gms of EO were fed to the reactor. Following the completion of the EO feed, the process was held at 125° C. to completely post-react all of the EO into the polyol over a period of about 3.5 hours. Then about 27 grams of 88% aqueous lactic acid were added to the reaction mixture to neutralize the polyol. About 4 grams of the stabilizer commercially available under the name Irganox™ 1076 were added to inhibit unintended oxidative degradation of the polyol during its handling and storage.

The resulting polyol, designated “NOP A” herein, was dark in color but transparent. This polyol had a hydroxyl number of about 193 mg KOH/g, a viscosity at 25° C. of 270 mPa·sec, a viscosity at 40° C. of 119 mPa·sec, with a pH measured at 7.4 and a moisture content of 0.05%.

### Example 2

### Preparation of NOP B (Comparative)

The KOH catalyst used to produce the polyol in accordance with this Example was used in an essentially anhydrous form by means of using the potassium salt of propoxylated glycerine (potassium alkoxide). The potassium alkoxide was made by charging 9620 grams of glycerine and 311 grams of aqueous potassium hydroxide (45%) into a 5-gallon stainless steel pressure-rated alkoxylation reactor at ambient temperature. The reactor was purged with nitrogen, closed, and heated to 110° C. Steady and thorough stirring of the liquid phase was applied, and vacuum was applied to the vapor space. Water vapor was condensed external to the reactor and the vacuum was discontinued after a period of one-two hours when the rate of water being condensed had greatly diminished. Vacuum was discontinued and the reactor was sealed in preparation for feeding the propylene oxide.

Propylene oxide was fed to the reactor gradually while the temperature of the liquid phase was maintained at 115° C. The total amount of propylene oxide fed to the reactor was 8110 grams over a time period of 5.5 hours. The propylene oxide (PO) was post-reacted completely by monitoring the pressure profile at 115° C. (isothermal conditions). The time required for post-reaction was 7 hours. The product was then vacuum stripped to assure that there would be no residual unreacted PO, and then it was cooled under a nitrogen blanket.

The result was a potassium alkoxide having a hydroxyl number of 977 mg KOH/gm, and potassium hydroxide concentration of 0.82% (as pure KOH) as determined by titration.

Preparation of a Short Chain NOP Polyether with a High Renewable Content:

3626 grams of the potassium alkoxide produced as described above and 8000 grams of soybean oil (refined, bleached, de-odorized; commercially available as “Cargill AR”) were charged at room temperature to a 5-gallon stainless steel pressure-rated alkoxylation reactor under a “nitrogen sweep”. The reactor, now free of air, was closed and pressurized with nitrogen.

The reactor contents were then heated to 125° C. and a nitrogen pressure of 1.1 bar, absolute was established in the reactor.

Ethylene oxide (EO) was fed to the reactor gradually while the temperature of the liquid phase was maintained at 125° C. The feed rate of EO was controlled by a feedback loop control to maintain a safe and relatively constant pressure.

6000 grams of ethylene oxide were fed to the reactor in 210 minutes. The reaction mixture was post-reacted at 125 to 130° C. until the pressure had decreased to a stable value indicating that all of the EO had reacted. The reaction mixture was cooled to 90° C. and 56 grams of 88% aqueous lactic acid were added to fully neutralize the residual alkalinity of the polyol. The mixture was then heated back up to 110° C. and full vacuum was applied to the vapor space of the reactor to remove moisture from the product.

9 grams of the anti-oxidant compound available under the name “Irganox 1076” were added to the reaction mixture. This corresponded to 500 ppm in the final product.

The mixture was then thoroughly mixed and cooled. The product was discharged from the reactor under a nitrogen blanket.

The polyether product was a clear liquid with a uniform appearance having the following properties:

| | |
|---|---|
| Hydroxyl Number (mg KOH/gm) | 210 |
| Acid Number (mg KOH/gm) | 0.024 |
| Water (%) | 0.015 |
| Color (Gardner) | 3 |
| pH (isopropanol/water) | 8.5 |
| Molecular weight distribution (Polydispersity) | 1.15 |
| Molecular weight, average (GPC) | 625 |
| Peak molecular weight (GPC) | 748 |

By theoretical calculation, the mean hydroxyl functionality of this polyether was estimated to be 2.1. By theoretical mass balance, this base polyol had a renewables content of 57%.

The following materials were used to produce the rigid polyurethane foams described in Examples 3-5:
NOP A: A KOH-catalyzed, ortho-TDA/soybean oil-initiated polyether polyol (100% EO) having a vegetable oil content of about 67%, a hydroxyl number of about 193 mg KOH/gm, a viscosity at 25° C. of about 270 mPa·sec, and a functionality of about 2.2.
NOP B: A KOH-catalyzed, glycerine/soybean oil-initiated polyether polyol (100% EO) having a vegetable oil content of about 45%, a hydroxyl number of about 210 mg KOH/gm, a viscosity at 25° C. of about 147 mPa·sec, and a functionality of about 2.1.
POLYOL C: A polyester polyol with a hydroxyl number of 240 mg KOH/g and a viscosity of about 3000 centipoise at 25° C., available from the Stepan Company as Stepanpol PS 2412.
POLYOL D: A sucrose based polyether polyol with a hydroxyl number of 470 mg KOH/g and a viscosity of about 33,000 centipoise at 25° C., available from Bayer MaterialScience as Multranol 4034.
FR 1: Tris(2-chloroispropyl) phosphate, a flame retardant available under the name Fyrol PCF from ICL Industrial Products.
FR 2: The diol of tetrabromophthalic acid, available from Albermarle as Saytex RB-79 Flame Retardant.
SURF: A silicon surfactant available from Evonik under the name Tegostab B-8465.
CAT 1: A polyisocyanurate catalyst available from Air Products as Dabco K-15 consisting of potassium octoate in diethylene glycol.
CAT 2: A polyurethane catalyst commercially available from Air Products as Polycat 43.
CAT 3: A catalyst available from Air Products as Polycat 5 consisting of pentamethyl diethylenetriamine.
Pentane: A 70:30 blend of cyclopentane: isopentane which is commercially available from Exxon Corporation under the name Exxsol 1600.
ISO: A polymeric methylene diphenyl diisocyanate available from Bayer MaterialScience under the name Mondur 489, having a viscosity of about 700 mPa·sec at 25° F. and an NCO content of about 30.6% mg KOH/g.

### Examples 3-5

Three different polyurethane foams were produced from the materials listed in Table 1 using the amounts indicated in Table 1. The physical properties of these foams are reported in Table 1.

TABLE 1

| Example | 3* | 4* | 5 |
|---|---|---|---|
| NOP A (pbw) | — | — | 46.12 |
| NOP B (pbw) | — | 46.12 | — |
| POLYOL C (pbw) | 46.98 | — | — |
| POLYOL D (pbw) | 15.56 | 15.37 | 15.37 |
| FR 1 (pbw) | 13.23 | 12.94 | 12.94 |
| FR 2 (pbw) | 3.23 | 3.20 | 3.20 |
| SURF (pbw) | 2.15 | 2.13 | 2.13 |
| CAT 1 (pbw) | 1.61 | 1.46 | 1.46 |
| CAT 2 (pbw) | 0.76 | 0.71 | 0.71 |
| CAT 3 (pbw) | 0.21 | 0.19 | 0.19 |
| Water (pbw) | 0.32 | 0.28 | 0.28 |
| Pentane (pbw) | 15.95 | 17.60 | 17.60 |
| ISO (pbw) | 152.5 | 141.7 | 141.7 |
| Gel Time (sec) | 40 | 38 | 39 |
| Overall Foam | 2.91 | 3.38 | 3.05 |
| Density (lbs/ft.³) | | | |
| Core Foam | 2.71 | 3.19 | 2.52 |
| Density (lbs/ft.³) | | | |
| k-Factor @ 75° F. | 0.161 | 0.161 | 0.150 |
| (BTU-in/h-ft.²-° F.) | | | |
| Dimensional | | | |
| Stability @ 7 days | | | |
| (% volume change) | | | |
| At −30° C. | 0.2 | −0.2 | −0.2 |
| At 70° C. | 0.8 | −1.0 | −0.1 |
| At 70° C./100% | 2.5 | 0.2 | 0.5 |
| Relative Humidity | | | |
| Open Cell | 8.8 | 13.0 | 11.7 |
| Content (%) | | | |

*Comparative Example

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of an amine-based polyol comprising reacting:
a) an amine adduct comprising the reaction product of
(i) an amine and
(ii) an alkylene oxide
in amounts such that from 1 to 2 moles of alkylene oxide are present for each amine group,
and
b) a triglyceride,
and optionally,
c) a compound containing at least one saccharose group and/or a glycerol,
in the presence of
d) an alkylene oxide,
and
e) a catalyst
to form an amine-based polyol characterized by a functionality of from about 1.5 to about 3.5, an equivalent weight of from about 100 to about 600, and an overall renewable content of from 20 to 85% by weight.

2. The process of claim 1 in which sucrose is used as c).

3. The process of claim 1 in which e) is potassium hydroxide (KOH) or a potassium alkoxide (KOR).

4. The process of claim 3 in which the KOH is used in an amount such that the final concentration of KOH is from 0.05 to 0.5% by weight, based on total weight of reaction mixture.

5. The process of claim 4 in which the KOH is neutralized after the amine-based polyol forming reaction has been completed.

6. The process of claim 1 in which the amine is toluene diamine.

7. The process of claim 1 in which the amine comprises 2,3-diamino toluene, 3,4-diamino toluene or a mixture thereof.

8. The process of claim 1 in which the triglyceride is selected from the group consisting of soybean oil, palm oil, palm kernel oil, castor oil, canola oil, high erucic acid content rapeseed oil, rapeseed oil, corn oil, jatropha oil, peanut oil, cottonseed oil, linseed oil, lard, tallow, bodied soybean oil, epoxidized soybean oil, camelina oil, lipids derived from algae, lesquerella oil, limnanthes oil, and mixtures thereof.

9. The process of claim 1 in which the alkylene oxide is selected from ethylene oxide, propylene oxide, butylene oxide and mixtures thereof.

10. The process of claim 1 in which ortho-toluene diamine adduct, the triglyceride and ethylene oxide are reacted to form the amine-based polyol.

11. The process of claim 10 in which the amine-based polyol is subsequently reacted with propylene oxide.

12. An amine-based polyol produced by the process of claim 1.

13. An amine/triglyceride-initiated polyether polyol characterized by a biorenewable content in the range of 50 to 85% by weight and an equivalent weight of from about 100 to about 600.

14. An o-TDA/soybean oil-initiated polyether polyol characterized by a biorenewable content in the range of from 50 to 85% by weight and an equivalent weight of from about 100 to about 600.

15. A process for the production of a rigid polyurethane foam comprising reacting:
a) an isocyanate-reactive component comprising an amine-based polyol produced by the process of claim 1,
b) an organic polyisocyanate, and
c) a blowing agent.

16. The process of claim 15 in which the blowing agent comprises water and a pentane.

17. A rigid polyurethane foam having a k-factor at 75° F. of from about 0.140 to about 0.160 BTU-in/h-ft.²° F. comprising the reaction product of
a) an isocyanate-reactive component comprising the amine-based polyol of claim 12,
b) an organic polyisocyanate, and
c) a blowing agent.

18. A rigid polyurethane foam having a k-factor at 75° F. of from about 0.140 to about 0.160 BTU-in/h-ft.²° F. comprising the reaction product of
a) an isocyanate-reactive component comprising the amine-based polyol of claim 13,
b) an organic polyisocyanate, and
c) a blowing agent.

19. A rigid polyurethane foam having a k-factor at 75° F. of from about 0.140 to about 0.160 BTU-in/h-ft.²° F. comprising the reaction product of
a) an isocyanate-reactive component comprising the amine-based polyol of claim 14,
b) an organic polyisocyanate, and
c) a blowing agent.

20. A rigid polyurethane foam having a k-factor at 75° F. of about 0.150 BTU-in/h-ft.²° F. comprising the reaction product of
a) an isocyanate-reactive component comprising the amine-based polyol of claim 12,
b) an organic polyisocyanate, and
c) a blowing agent.

21. A rigid polyurethane foam having a k-factor at 75° F. of about 0.150 BTU-in/h-ft.²° F. comprising the reaction product of
a) an isocyanate-reactive component comprising the amine-based polyol of claim 13,
b) an organic polyisocyanate, and
c) a blowing agent.

22. A rigid polyurethane foam having a k-factor at 75° F. of about 0.150 BTU-in/h-ft.²° F. comprising the reaction product of
a) an isocyanate-reactive component comprising the amine-based polyol of claim 14,
b) an organic polyisocyanate, and
c) a blowing agent.
